(19) United States
(12) Patent Application Publication
(10) Pub. No.: US 2018/0168521 A1
(43) Pub. Date: Jun. 21, 2018

(54) KNEE POSITIONER

(71) Applicant: BRAINCON Handels-GmbH, Wien (AT)

(72) Inventors: Stefan Nehrer, Wien (AT); Davul Ljuhar, Wien (AT)

(21) Appl. No.: 15/552,147

(22) PCT Filed: Feb. 18, 2016
(86) PCT No.: PCT/AT2016/050032

§ 371 Date: Aug. 18, 2017

(30) Foreign Application Priority Data
Feb. 19, 2015 (AT) A92/2015

(51) Int. Cl. A61B 6/04; A61B 6/00

(52) U.S. Cl. CPC A61B 6/04; G03B 42/025; A61B 6/4283

(57) ABSTRACT

The device according to the disclosure is for stabilising a patient in a desired position during X-ray knee imaging. By default, a tibial inclination angle of 10° is envisaged, as according to statistics this constitutes an inclination for the ideal imaging of the knee joint space. This angle as well as the foot rotation angle are adjustable. The device is suitable for a majority of different body heights and shapes. Equipment sensors determine the achievement of the desired position and this is confirmed to the X-ray technician by means of an acoustic and optical transducer.

### CROSS-REFERENCE TO RELATED APPLICATIONS

This application is a National Stage application of International Application No. PCT/AT2016/050032, filed Feb. 18, 2016, which claims benefit to Austrian Application No. A92/2015, filed Feb. 19, 2015, which are incorporated by reference herein in their entities.

### BACKGROUND

### Field

The invention relates to a device for precisely, reproducibly positioning a test subject's tibia and knee joint to be diagnosed radiologically on a sagittal, transversal and frontal plane.

### Description of the Related Art

Many X-ray procedures of the knee are performed with the subject standing in an upright position, because legs and knees will then have to carry the subject's body weight, and it is only then that rational medical assessments, e.g., of the knee joint space for evaluating arthroses, can be performed.

In doing so, the patient will be instructed to adopt the appropriate posture and position, and the image will be taken. However, it is nearly impossible for the subject to self-supportingly hold their knee in one exact position and remain perfectly still at the same time.

Moreover, studies have shown that the human tibia is sloping downwards from its joint head towards the femur and the back of the knee, respectively (postural tibial plateau slope).

On average, this descending slope has a mean angle of 10°. Therefore, it would be advisable to X-ray the knee in a position which is tilted forwards in an angle of 10° (see angle a in FIG. 5), in order to ideally visualise the knee joint space (X-ray axis is parallel to the knee joint space). This is, however, commonly neglected in the field.

Such reasons result in knee joint space analyses having a very high measuring inaccuracy, as even the slightest change in perspective usually has a serious impact on the knee joint space measuring value. Also contributing to this are minimal movement artefacts that cannot be avoided while a subject is self-supported, which lead to a more or less pronounced blurring of the X-ray image resulting in an aggravated visual evaluation as well as by automated algorithms.

Various products addressing these problems have already been developed. The “holding device” by Telos is a stabilising device for recumbent imaging. It offers a variety of possibilities for stabilising different body parts in certain positions. Three fixation points guarantee good stability. This device can however only be used in recumbent imaging. Especially the assessment of the knee joint space, however, requires vertical imaging, because it will be compressed under stress and can thus be reproducibly analysed. In a recumbent position, the height of the knee joint space can vary greatly depending on the elasticity of the subject's ligaments.

Another product striving for standardised knee imaging is the SynaFlexer by Synarc. It is intended for horizontal imaging and consists of an acrylic glass form to be slid in front of the detector. During imaging, the patient's knee is resting against the acrylic glass wall. In doing so, the patient automatically adopts a position with a forward tibial inclination angle. Additionally, X-rays will be adjusted accordingly to reach a downwardly sloping angle of 10°. This will however lead to a distortion at the detector which remains vertical. Furthermore, studies have already shown that by default the human tibial plateau is backwardly sloping with an angle of 10°. In order to obtain ideal images for assessing the knee joint space, the X-ray axis has to run parallel thereto, which is not the case with the SynaFlexer. Additionally, there is a stabilising splint following the centre of the acrylic glass wall, which is visible on the X-ray image.

### SUMMARY

It is the object of this invention to ensure that the joint is evaluated in a standardised secured position during X-raying, and thus to markedly increase repeatability and evaluating quality.

According to the invention, this is achieved by providing a guide through which a tibial inclination angle is settable, and by providing at least two equipment sensors simultaneously activatable by the anterior tibial crest at full contact, so that the tibial inclination angle set by the guide is monitored via the two equipment sensors, and by providing at least one pivotable foot panel through which the rotation of the foot is settable.

Further embodiments of the invention are described herein below.

### BRIEF DESCRIPTION OF THE DRAWINGS

FIG. 1 shows a diagram of the repeatability of the tibial inclination angle with and without the device according to the invention;

FIG. 2 is a top view of a base of one embodiment of the device according to the invention;

FIGS. 3 and 4 are each angled views of a foot panel belonging to said base according to FIG. 2, and

FIG. 5 is a schematic lateral view of a projection of an X-ray device while maintaining a fixed tibial inclination angle.

The device according to the invention has been developed in order to help the patient to steadily stand in an ideal position during knee X-rays.

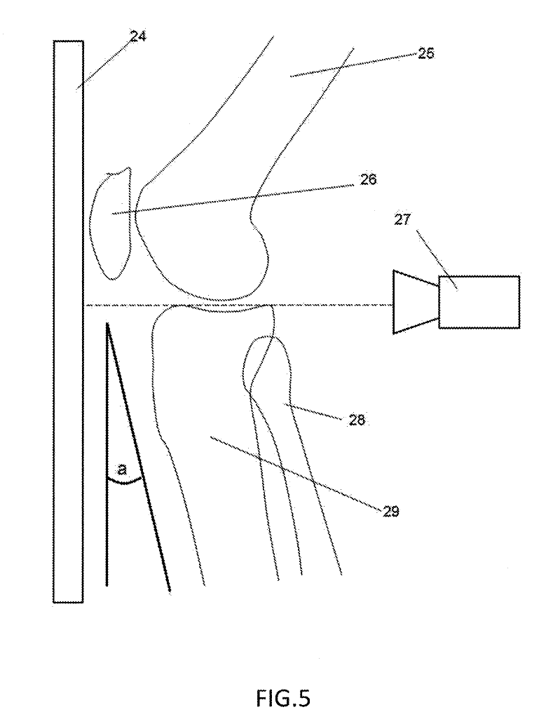

In FIG. 5, an X-ray beam is emerging from an X-ray tube 27 passing through a knee joint space and visualising it on a receiving element 24. Femur 25 with knee cap 26 and tibia 29 are visualised in vertical positioning.

### DETAILED DESCRIPTION

By default, the ideal position requires a tibial inclination angle of 10° (see a in FIG. 5). Depending on the markedness of the patient's tibial plateau however, said angle can be gradually adjusted. Additionally, a rotation of the joint is prevented by securing same.

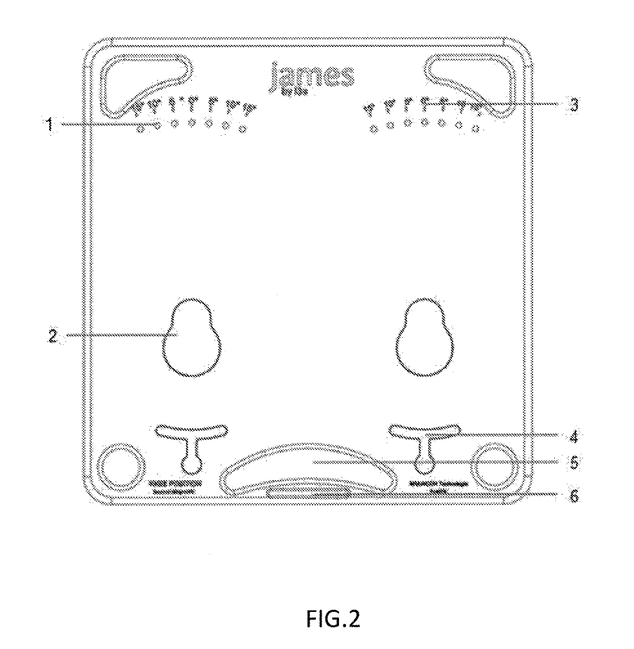

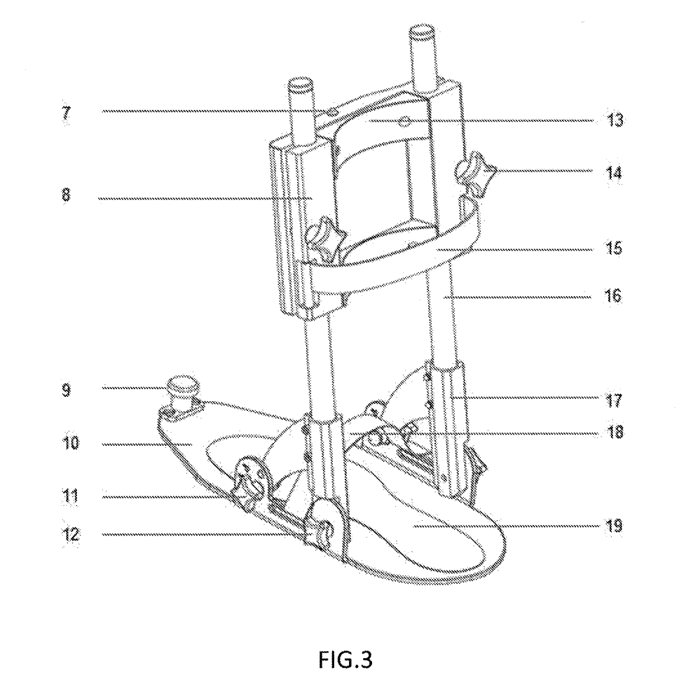

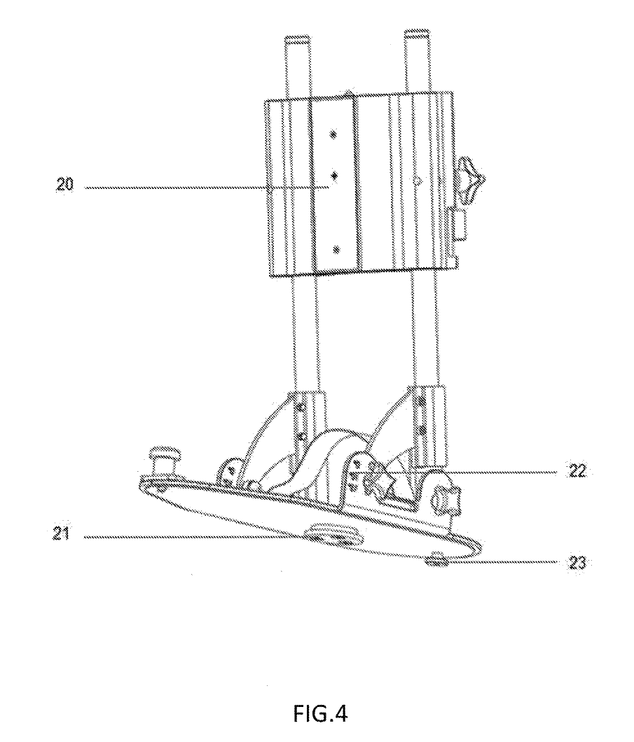

A securing structure comprises a metal bottom plate (FIG. 2) as a base having latching and docking recesses in the form of a hat-slot pin 2, 3, 4 for each one of the right and left foot panels. The foot panels (FIG. 3, FIG. 4) can be inserted into said recesses and aligned. Said foot panels—one each for the left and right bases—consist of a foot plate 10 which can be docked into the bottom plate (FIG. 2), a foot loop 18 for securing the foot, two guide rods 16 and one support element 8 attached thereto, including a securing loop 15 for the lower leg.

Said foot plates 10 can also accommodate very big foot sizes and can incrementally be rotated, in order to correspond to the individual outside rotation of the patient's feet. Angles from −20° to 20° are adjustable in steps, e.g., of 5°.

The foot loops 18 are directly attached to the foot plates 10, and are variably securable by means of a hook and loop fastener without making the patient feel uncomfortable. In order to be able to adjust the guide rod 16 inclination and ensuring their stability, they are dually fixed to the foot plates 10, the posterior attachment 12 being permanent and the anterior attachment 11 being incrementally adjustable, in order to change the inclination angle. Settable angles include e.g., 0°, 10° and 15 (22).

The device elements 8 are directly attached to the guide rods 16 and their height can be steplessly adjusted via clamping screws 14 in order to accommodate a great variety of body heights. Pressure sensors 13 are included in the device elements as equipment sensors. If pressure is applied to such sensors, they actuate two other elements of the module as a transducer, e.g., an acoustic signal, as well as an optical colour signal by LEDs.

As a result, when the anterior tibial crest is positioned correctly with the appropriate angle prescribed by the support modules, feedback is provided. Pitch levels and illuminating colours are chosen to be differently expressed for each side of the body, in order to be able to discern if both or only one leg is/are positioned correctly, or not.

Optionally, a switch supporting an X-ray release mechanism will be activated.

Construction elements positioned within the range of X-rays, i.e. support elements 8 and guide rods 16, consist of X-ray-transparent materials, such as GRP, CRP or plastics such as PP, PEEK, etc.

For both lower legs, securing loops 15 are embodied as hook and loop fasteners, similar to those securing the feet.

Owing to scientific evaluations it was demonstrated that repeatability will markedly increase by using said knee positioner and reach an extent allowing for reliable joint progress analyses. For this purpose, AP knee X-rays were taken of patients with the following parameters: a) self-supporting and ordered to slightly bend their knees in order achieve a tibial inclination of approximately 10° and the knee cap contacting the detector; b) using a knee positioner with a tibial inclination of 10° while ordered to uniformly distribute their weight via the tibia onto the upper and lower panels of the support modules; c) using a knee positioner with a tibial inclination of 10° while ordered to uniformly distribute their weight via the tibia onto the upper and lower panels of the support modules until the support sensors are triggered.

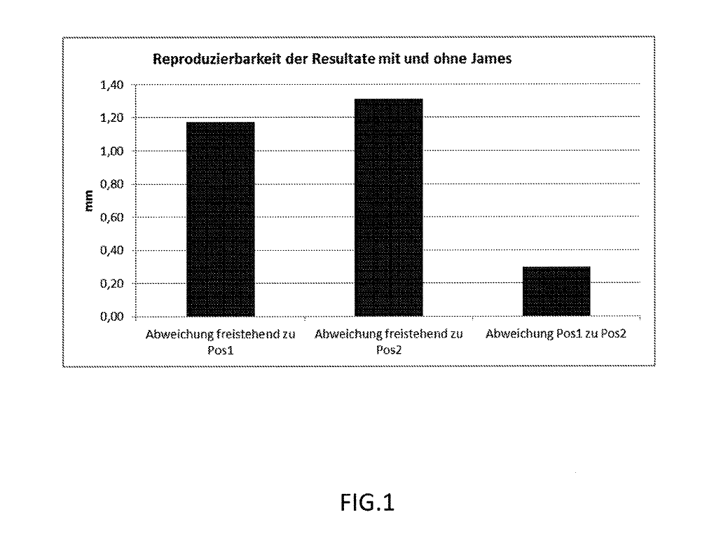

Using a software identifying the corresponding contours automatically and reproducibly (i3a JSX), the measured height of the medial and lateral knee joint space was evaluated in millimetres. Using the device according to this invention, a deviation lower than 1% could be achieved. Without providing feedback regarding the correct positioning, deviations reach approximately 10%, and in a self-supporting position, deviations of approximately 30% can be expected. See, also FIG. 1.

What is claimed is:

1. A device for precisely, reproducibly positioning a test subject's tibia and knee joint to be diagnosed radiologically in a sagittal, transversal as well as frontal plane, the device comprising:
a guide through which a tibial inclination angle is settable;
at least two equipment sensors which are simultaneously activatable at full contact by the anterior tibial crest, so that said tibial inclination angle predefined by said guide is monitored by the two equipment sensors; and
at least one pivotable foot panel through which the foot's rotation is predefinable.

2. The device according to claim 1, wherein the equipment sensors are formed by mechanical or electromechanical sensors, limit switches, pressure sensors, inductive, resistive or capacitive proximity switches or as a light barrier.

3. The device according to claim 1, wherein, upon correct positioning of the test subject, the device is providing feedback through one or more transducers in the form of light, noise etc. perceivable by radiology staff or triggering a switching contact for releasing the X-ray image.

4. The device according to claim 3, wherein the signal(s) for positioning the left and right legs differ from each other in terms of interval (flashing/noise timing) and/or frequency (light colour/pitch).

5. The device according to claim 4, wherein said device is adjustable to various body shapes of the test subject, the tibial inclination angle being settable within a range of from 0° to 25°, and the foot rotation being settable within a range of from −20° to +20°.

6. The device according to claim 1, further comprising:
a base; and
a left and a right pivotable foot panel.

7. The device according to claim 6, wherein the foot panels are pivotably attachable to the base through one or more hat-slot pins and corresponding recesses therein.

8. The device according to claim 6, wherein the foot panels each comprise a foot loop for securing the foot, and the guide is formed by two laterally arranged guide rods and a support element attached thereto having a securing loop for the lower leg.

9. The device according to claim 8, wherein the guide rods for adjusting the tibial inclination angle are pivotably arranged transversely to the longitudinal foot axis.

10. The device according to claim 8, wherein the equipment sensors are formed as pressure sensors which are integrated into the support elements.

11. The device according to claim 1, wherein assemblies, especially said guide rods and support elements, which are arranged within the range of the X-ray, are embodied in X-ray-transparent materials.

12. The device according to claim 1, wherein an X-ray beam of an X-ray device, for posteroanterior (PA) X-ray imaging, is entering into the side of the back of the knee and emerging from the side of the knee cap, where it impinges onto a detector of the X-ray device.
